(19) United States
(12) Patent Application Publication
(10) Pub. No.: US 2016/0069910 A1
(43) Pub. Date: Mar. 10, 2016

(54) DETECTING ENDOCRINE DISRUPTING COMPOUNDS

(71) Applicant: STELLENBOSCH UNIVERSITY, Stellenbosch (ZA)

(72) Inventors: Pieter SWART, Stellenbosch (ZA); Barry Eric TRUEBODY, Somerset West (ZA)

(73) Assignee: STELLENBOSCH UNIVERSITY, Stellenbosch (ZA)

(21) Appl. No.: 14/932,350

(22) Filed: Nov. 4, 2015

Related U.S. Application Data
(63) Continuation of application No. PCT/IB2014/061242, filed on May 6, 2014.

(30) Foreign Application Priority Data
May 6, 2013 (ZA) 2013/03249

(51) Int. Cl. G01N 33/74

(52) U.S. Cl. CPC G01N 33/743; G01N 2333/723

(57) ABSTRACT

A method and a detection device for detecting endocrine disrupting compounds are provided. The detection device includes a support structure and ligand binding domains (LBD) of at least one sex hormone receptor, immobilised on the support structure. The detection device further includes a linker molecule between the support structure and the ligand binding domains, for immobilising the LBD's on the support structure.

This disclosure relates to detecting endocrine disrupting compounds. In particular the disclosure relates to a detection device for detecting endocrine disrupting compounds and a method of detecting endocrine disrupting compounds.

### BACKGROUND

It is well known that certain hormone active agents in the environment can disrupt chemical messengers (hormones) of the endocrine system by sending erroneous signals or blocking legitimate signals. The putative hormone active agents, also known as endocrine disrupting compounds (EDCs), exert their deleterious effects on humans and wildlife by mimicking, blocking and disrupting the physiological functions of hormones. The Endocrine Society of America brought out a comprehensive report in 2009 presenting evidence that endocrine disruptors effect male and female reproduction, breast development and cancer, prostate cancer, neuroendocrinology, thyroid activity, metabolism, obesity and cardiovascular endocrinology (Diamanti-Kandarakis et al., 2009). The report concludes that results from animal models, human clinical observations, and epidemiological studies supply overwhelming evidence that EDCs pose a significant concern to public health.

Hormones exert their functions by interacting with their corresponding receptors in target cells to trigger responses and prompt normal biological functions such as growth, development, behaviour and reproduction. Interference with the activities of hormones, such as is the case with EDCs, can lead to reversible or irreversible abnormal biological outcomes including stunted growth, impairment of short term memory, tubal pregnancy, low sperm count, reproductive failure and damage of the immune system. It is clear that as researchers continue to look at the adverse effects caused by these hazardous compounds on humans and wildlife, they continue to find significant, often permanent, effects at remarkably low doses.

Endocrine disrupting compounds can be categorized into three major groups: androgenic (compounds that mimic or block natural testosterone), thyroidal (compounds with a direct or indirect impact on the thyroid glands) and estrogenic (compounds that mimic or block natural estrogens). Despite the broad spectrum of EDCs, the estrogenic compounds (ECs) are the most prominent and studied of these classes of compounds. This is in all likelihood due to the importance of ECs in cancer research, specifically related to female sexual development and disorders (McLachlan et al., 2006; Darbre and Charles, 2010; Fenton, 2006; Newbold et al., 2007). Estrogenic compounds are found in low doses in literally thousands of products, and include compounds like diethylstilbestrol, bisphenol A, polybrominated diphenyl ethers and phthalates. Androgenic EDCs have also attracted much attention due to their effects on male virility and fertility (Bay, Asklund et al., 2006; Sharpe, 2006; Skakkebak et al., 2001). Endocrine disrupting compounds have been widely reported to be present in very low concentrations in the environment, but their relatively high fat solubility causes these substances to bio-accumulate in fat deposits of organisms and animals higher up in the food chain, leading to significant physiological responses at these relatively low concentrations. Other relevant sources of EDCs are found in insecticides, herbicides, fungicides, plasticizers, plastics, resins and industrial chemicals such as detergents (Diamanti-Kandarakis et al., 2009). The hydrophobicity of EDCs, coupled with other chemical properties, has created unique challenges for environmental analytical chemists in developing techniques required for detecting and screening them. Several analytical techniques have been used. These methods frequently include solid phase extraction (SPE) followed by: high performance liquid chromatography (HPLC), liquid chromatography/mass spectrophotometry (LC/MS) or gas chromatography/mass spectrophotometry (GC/MS). These techniques are, however, limited for general EDC monitoring due to relative high instrument costs, intensive labour and, in some instances, relatively poor sensitivity. In addition high-end analytical procedures are usually specific for one single analyte only, or a limited class of structurally related compounds.

Affinity chromatography (AC) is a powerful chromatographic technique which utilises the specific interaction between a biological molecule and a ligand (hormone receptor and hormone) to affect the specific binding and isolation of a ligand or substrate analogue (Cuatrecasas, 1970). During AC bio-specific and reversible interactions are used for the selective separation and purification of biological molecules from complex biological matrices. These systems are increasingly applied in the field of biotechnology due to the ability of the technique to specifically bind and remove bio-molecules from complex mixtures. Typical affinity systems consist of two distinct parts: the mobile phase, which carries the biological molecule to be separated, and the solid phase, which is usually modified to carry the affinity ligands. Notwithstanding the fact that these systems are widely used, they still have some shortcomings, including the requirements for a large column set-up and a longer diffusion path length, which in turn leads to a significant increase in the time required for the entire downstream processing from the introduction of the crude extract to the final purified product. Membrane affinity chromatography (MAC) was introduced to overcome the major shortcomings of column affinity chromatography. Its introduction has significantly reduced the number of steps needed to obtain a pure product due to the specificity of the interaction between the stationary phase and the target bio molecule, not withholding the larger surface area and shorter diffusion path length that the system offers. With the above-mentioned advantages affinity membrane systems (AMS) could serve as a powerful method for analytical detection, and possibly removal processes, for EDCs from the environment.

Estrogenic compounds are a class of EDCs which mimic or block the endogenous estrogen activity by binding to the ligand binding domain (hERαLBD) of estrogen receptors (ERs) in the endocrine system. Exploiting the interaction between estrogen and its receptors, and using the chemical information obtained from this interaction, a more reliable and specific analytical functionalized affinity membrane system for the initial capture, concentration and qualitative detection of ECs can be developed. This method will be supplemental and used in conjunction with high technology analytical techniques such as HPLC, GC/MS and LC/MS for detecting ECs in the environment. A similar system can also be used for the androgenic EDCs using the hARLBD.

The inventors identified a new detection device, which is rapid and efficient in monitoring and detecting endocrine disrupting compounds.

References in this specification to the term “sex hormone receptors” specifically refer to estrogen and androgen receptors.

In this specification the following abbreviations will be used:
³HE2 2, 4, 6, 7 ³H 17β-estradiol
AC Affinity chromatography
AMS Affinity membrane system
AR Androgen receptor
CA Cellulose acetate
CPM Counts per minute
DMDDO Dicarboxymethyl-3,6-diazaoctanedioate
DMF Dimethyl formamide
DMSO Dimethyl sulphoxide
E2 17β-estradiol
EC Estrogenic compound
EDC Endocrine disrupting compound
EDTA Ethyl diamine tetraacetic acid
ELISA Enzyme linked immunosorbent assay
ELRA Enzyme linked receptor assay

ER Estrogen receptor
ERE Estrogen response element
FT-IR Fourier Transformed infrared spectroscopy
GC Gas chromatography
GC/MS Gas chromatography/Mass spectrophotometry
HF Hollow fibre
HFF Hollow fine fibre
his₆ Hexahistidine
HPLC High performance liquid chromatography
HRP Horseradish peroxidase
IMAC Immobilized metal ion affinity chromatography
LBD Ligand binding domain
LC/MS Liquid chromatography/Mass spectrophotometry
LLD Lowest limit of detection
MAC Membrane affinity chromatography
MBP Maltose binding protein
MS Mass spectrophotometry
NMR Nuclear magnetic resonance
PCR Polymerase chain reaction
PDMDDO Pluronic-N,N-dicarboxymethyl-3,6-diazaoctanedioate
PEI Polyether imide
PIXES Proton induced x-ray emission spectrophotometry
PS Polystyrene
PSU Polysulphone
PVDF Polyvinyldiene fluoride
SDS PAGE Sodium dodecyl sulphate polyacrylamide gel electrophoresis
SEM Scanning electron microscopy
SPE Solid phase extraction
UF Ultrafiltration

In this specification reference will be made to the following documents:

BAY, K., ASKLUND, C., SKAKKEBAEK, N. E. and ANDERSSON, A. M., 2006. Testicular dysgenesis syndrome: possible role of endocrine disrupters. Best Practice & Research in Clinical Endocrinology & Metabolism, 20(1), 77-90.

CUATRECASAS, P., 1970. Protein purification by affinity chromatography. Journal of Biological Chemistry, 245(12), 3059-3065.

DARBRE, P. D. and CHARLES, A. K., 2010. Environmental oestrogens and breast cancer: evidence for combined involvement of dietary, household and cosmetic xenoestrogens. Anticancer Research, 30(3), 815-827.

DIAMANTI-KANDARAKIS, E., BOURGUIGNON, J. P., GIUDICE, L. C., HAUSER, R., PRINS, G. S., SOTO, A. M., ZOELLER, R. T. and GORE, A. C., 2009. Endocrine-disrupting chemicals: an Endocrine Society scientific statement. Endocrine Reviews, 30(4), 293-342.

FENTON, S. E., 2006. Endocrine-disrupting compounds and mammary gland development: early exposure and later life consequences. Endocrinology 147(6) (Supplement):S18-S24.

GOVENDER, S., PRZYBYLOWICZ, W., JACOBS, E., BREDENKAMP, M., KRALINGEN, L. and SWART, P., 2006. A Pluronic-coupled metal-chelating ligand for membrane affinity chromatography. Journal of Membrane Science, 279(1-2), 120-128.

MCLACHLAN, J. A., SIMPSON, E. and MARTIN, M., 2006. Endocrine disrupters and female reproductive health. Best Practice & Research Clinical Endocrinology & Metabolism, 20(1), 63-75.

NEWBOLD, R. R., JEFFERSON, W. N. and PADILLA-BANKS, E., 2007. Long-term adverse effects of neonatal exposure to bisphenol A on the murine female reproductive tract. Reproductive Toxicology, 24(2), 253-258.

SHARPE, R. M., 2006. Pathways of endocrine disruption during male sexual differentiation and masculinisation. Best Practice & Research Clinical Endocrinology & Metabolism, 20(1), 91-110.

SKAKKEBAK, N., RAJPERT-DE MEYTS, E. and MAIN, K., 2001. Testicular dysgenesis syndrome: an increasingly common developmental disorder with environmental aspects: Opinion. Human Reproduction, 16(5), 972-978.

### SUMMARY OF THE INVENTION

According to one embodiment of the invention, there is provided a detection device for detecting endocrine disrupting compounds, which includes
a support structure; and
ligand binding domains (LBD) of at least one sex hormone receptor, immobilised on the support structure.

The detection device may include a linker molecule between the support structure and the ligand binding domain.

The endocrine disrupting compounds may be in the form of any one or more of androgenic and estrogenic compounds.

The sex hormone receptor may be in the form of any one or more of an estrogen receptor and an androgen receptor. Specifically the sex hormone receptor may be in the form of any one or more of a human estrogen receptor and a human androgen receptor, having ligand binding domains hERαLBD and hARLBD respectively. The ligand binding domain may include a peptide tag and be in the form of his₆-hERαLBD or his₆-hARLBD. Alternatively the ligand binding domain may be expressed as a fusion protein and be in the form of maltose binding protein-hERαLBD (MBP-hERαLBD).

The ligand binding domain of the sex hormone receptor, may be prepared by cloning the genes encoding the ligand binding domain and expressing the genes in a host. Preferably the host may be Escherichia coli.

The support structure may be in the form of a membrane. The support structure may be in the form of a membrane contactor (strip matrix).

In one embodiment the membrane contactor may be in the form of a cellulose acetate hybrid membrane, specifically an affinity cellulose acetate-amylose hybrid membrane. In such embodiment the linker molecule may be in the form of maltose binding protein.

In another embodiment the support structure may be in the form of an inert membrane. The inert membrane may be in the form of a synthetic polymeric membrane. The synthetic polymeric membrane may be fabricated using the immersion precipitation technique. The synthetic polymeric membrane may be planar and nonporous. The synthetic polymeric membrane may be cast from any one or more of polysulphone (PSU), polyether imide (PEI) and polyvinylidene fluoride (PVDF) solutions.

In such embodiment the linker molecule may be in the form of a modified poloxamer. The poloxamer may be in the form of a difunctional block copolymer surfactant terminating in primary hydroxyl groups. The poloxamer may be in the form of poly(ethylene glycol)-block-poly(propylene glycol)-block-poly(ethylene glycol) (hereafter referred to as Pluronic F108, also known as PEG-PPG-PEG).

The modified Pluronic F108 may be in the form of Pluronic F108 of which the hydroxyl end groups was modified to a metal chelating moiety which can specifically bind Ni-ions and serve as an immobilised metal affinity chromatography matrix and provides a metal affinity immobilised receptor ligand binding domain. Specifically an ethylene diamine tetraacetic acid dianhydride may be coupled to the terminal hydroxyl end groups of Pluronic F108 via a two-step reaction, to create the new metal affinity ligand, Pluronic-N,N dicarboxymethyl-3,6-diazaoctanediote (Pluronic-DMDDO).

Another aspect of the disclosed embodiments relate to a method of detecting endocrine disrupting compounds, which includes
contacting a detection device as described with a sample to be tested, such that if the sample contains endocrine disrupting compounds, the compounds will bind to the ligand binding domains; and
performing a colorimetric assay to indicate bound endocrine disrupting compounds.

The sample to be tested may be in the form of a water sample.

Contacting the detection device with the sample, may be by way of submerging the detection device in the sample, dropping the sample onto the detection device, dipping the detection device in the sample or the like.

In one embodiment performing the colorimetric assay may include the steps of
saturating the receptors with EDCs, upon which the receptors change conformation and are activated;
adding monoclonal antibodies, raised against the activated receptors, and coupled to enzymes, allowing the antibody-enzyme complex to bind to the activated bound receptor;
observing the bound enzymes, which indicates the presence of EDCs in the sample.

In this embodiment the enzyme may be in the form of an enzyme which produces a coloured, fluorimetric or luminescent derivative of the activated bound receptor. The enzyme may be in the form of horse radish peroxide.

In another embodiment performing the colorimetric assay may include the steps of
adding enzyme labelled steroid ligands for the specific receptors; and
determining the degree of receptor saturation.

In yet a further embodiment performing the colorimetric assay may include the steps of
adding heat shock proteins, which binds and activates bound receptors; and
adding antibodies against the heat shock proteins to indicate bound receptors and thus endocrine disrupting compound binding.

The various disclosed embodiments will now be described, by way of example only with reference to the following representation(s):

### DRAWING(S)

In the drawing(s):

FIG. 1 shows a schematic representation of the immobilized steroid ligand binding domain for the affinity capture of EDCs in drinking water;

FIG. 2 shows a schematic representation of a method of detection of bound EDCs.

### EMBODIMENT OF THE INVENTION

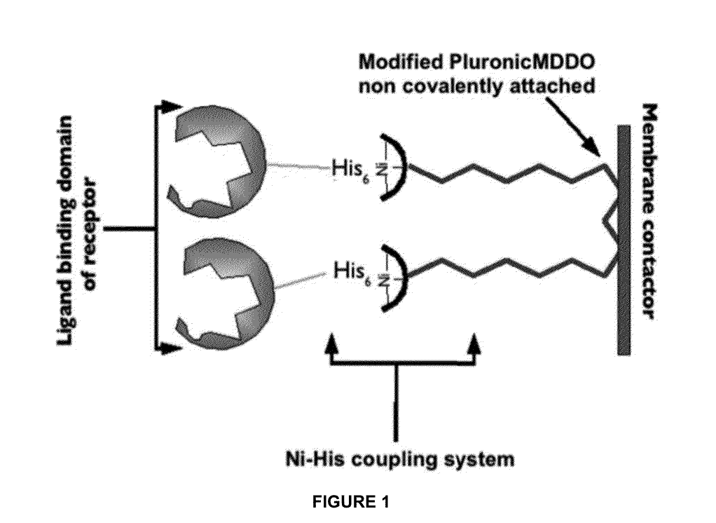

In FIG. 1 a schematic representation of a detection device for detecting endocrine disrupting compounds is shown. The detection device includes a support structure in the form of a membrane contactor and ligand binding domains (LBDs) of at least one sex hormone receptor, immobilised on the support structure. The detection device further includes a linker molecule, in the form of modified PluronicMDDO between the support structure and the ligand binding domain.

The detection device works on the following principles: The ligand binding domain of the hERαLBD and the hARLBD are immobilised on a membrane contactor matrix using noncovalent AC technology. The immobilised receptor is exposed to the water containing low concentrations of estrogenic and/or androgenic compounds. These compounds are bound to the corresponding receptor and concentrated on the contactor surface through the receptor ligand interaction.

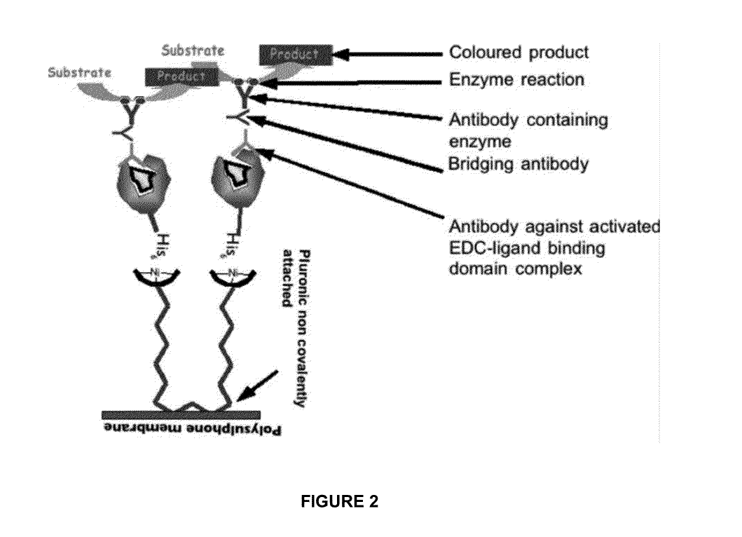

In FIG. 2 a method of detecting endocrine disrupting compounds is shown. The first step of this method entails contacting a detection device as described with a sample to be tested, such that if the sample contains endocrine disrupting compounds, the compounds will bind to the ligand binding domains. The second step is to perform a colorimetric assay to indicate bound endocrine disrupting compounds.

As shown in FIG. 2, after activation, a mild increase in temperature, the hERαLBD- or hARLBD estrogenic/androgenic compound-complex can be indicated using specific antibodies in an enzyme linked immuno-assay system. The presence of estrogenic/androgenic compounds will be indicated by the development of a specific colour on the contactor “strips”.

Methodology

Membrane Fabrication

Planar nonporous membranes were cast from appropriate solutions, polysulphone (PSU), polyether imide (PEI) and polyvinylidene fluoride (PVDF). The solutions were degassed before use to cast 200 μm planar membranes. Nonporous hollow fibre (HF) and hollow fine fibre (HFF) membranes and externally unskinned ultrafiltration (UF) membranes were produced by the phase inversion technique using a dry-wet spinning process available at the Institute for Polymer Science at the University of Stellenbosch.

An amylose functionalized cellulose acetate (CA) membrane was fabricated from a solution of CA and amylose in DMSO via immersion precipitation. The casting mixture was then used to cast flatsheet membranes by immersion precipitation using water. The membranes were thoroughly washed with water and stored in a sodium azide solution until required. The surface membrane topography, thickness and functional groups modification were determined using SEM and FT-IR respectively.

A Pluronic® F108 modified linker was synthesized as follows: The terminal hydroxy groups of Pluronic were modified in a two-step reaction to yield the tetra dentate DMDDO type ligand at the hydroxyl terminals of Pluronic. This reaction was carried out by dissolving a ten-fold excess of the EDTA dianhydride and imidazole dry DMF after which Pluronic F108 was added and reacted for 8 h at 40° C. Methanol was subsequently added and reacted for another 8 h after which the DMF was removed in vacuo. The residue was treated with toluene to selectively dissolve ligand-modified-Pluronic from the DMDDO by-product. The final product was characterised mainly by nuclear magnetic resonance (NMR) with the aid of model ligands based on mono and diethylene glycol (Govender et al., 2006).

Receptor Expression

The introduction of a peptide tag, or even low molecular weight proteins, to the N- or C-terminus of proteins has become a convenient and facile method for protein purification via affinity chromatography. Examples of tags include polyhistidine tags and maltose to produce fusion proteins. As the principle of non-covalent immobilization of the truncated steroid receptors forms the basis for the test strip envisaged in this project, the histidine tag allows for the purification of the receptors and a method for non-covalent immobilization following the principle of IMAC. Thus the ligand binding domain of the steroid receptors had to be subcloned into a suitable vector encoding for a histidine tag, the plasmid construct transformed into a suitable host organism and finally expressed as histidine tagged fusion proteins.

The gene encoding for a hexahistidine tagged hERαLBD, subcloned into pET15b (a plasmid that encodes for an N-terminal hexahistidine tag), was obtained from the University of Illinois at Urbana-Champaign. The hERαLBD protein was expressed in Escherichia coli BL21 (DE3) pLysS. The initial yield of the soluble protein compared to the insoluble protein, was insufficient. Experiments to improve the solubilisation of the hERαLBD protein prior to purification were performed. These included the use of the detergent Nonidet P-40 (NP-40), which increases protein solubility, during cell lysis. In addition, 17β-estradiol (E2) and sucrose were included during the expression experiments as previous studies showed that these additives increased the yield of soluble vs. insoluble protein. Protein detection methods (sodium dodecyl sulphate polyacrylamide gel electrophoresis (SDS PAGE) and Western Blot analysis) showed that the increase in soluble hERαLBD protein was achieved in the presence of 10 μM E2 (expression results in the presence of E2 showed a higher soluble protein when compared to the expression without E2).

Using the full length AR gene the truncated human androgen receptor ligand binding domain, hARLBD, was amplified via polymerase chain reaction (PCR) and subcloned into pTrcHis, a plasmid encoding for an N-terminal hexahistidine tag. The construct was designated AR-pTrcHis. The construct was subsequently used to transform TOP10 E. coli. Expression and purification of the hARLBD were identical to that of the hERαLBD with the exception of the absence of 17β-estradiol.

In this study the targeted protein hERαLBD was expressed as a fusion protein to maltose binding protein (MBP). Maltose binding protein, being the main supplier of carbon to gram-negative bacteria, is known to have a great affinity for maltose and, in the absence of the maltose, has a great affinity for amylose. The affinity chromatographic column used in this study was set up using amylose resin. Purification of the recombinant protein was possible, since these proteins will couple (using the MBP) to the amylose column via affinity interaction. The gene was ligated into a plasmid expression vector pMalc2 and transformed into E. coli TB1 cells for expression. The hERαLBD was expressed as a fusion protein to fusion partner MBP, a MalE gene of molecular mass 40.6 kDa.

Receptor Purification and Antibody Production Against Fusion Receptors

Preliminary purification of the hERαLBD protein with the use of a prepacked nickel agarose column (His SpinTrap, GE Healthcare) showed efficient binding of the histidine tagged hERαLBD protein to the resin. The protein was subsequently purified on a preparative AKTA® system. The hARLBD was purified under identical conditions to that of the hERαLBD. The MBP-hERαLBD fusion proteins and the MBP proteins were purified on an amylose column by affinity chromatography. The coupled fusion protein was eluted from the affinity column by including 10 mM of maltose in the elution buffer.

The purified hERαLBD, saturated with E2, was then inoculated into a rabbit in order to generate polyclonal antibodies against the E2-bound form of hERαLBD. Blood samples were drawn from the rabbit on days 0 and 28. The blood samples were stored at 4° C. and allowed to coagulate after which they were centrifuged, separating the serum from the coagulant. The serum was used as the source of antibodies.

Binding Studies

For estrogen binding studies, PVDF membranes and polystyrene microtitre plates (Immunosorp Nunc®) were used as a scaffold for the Pluronic-DMDDO. Microtitre plates, in addition to the hydrophobic nature of the polystyrene polymer from which they are constructed, are experimentally more practical, easier to handle and allow for greater throughput. Wells of a polystyrene plate were incubated with a solution of Pluronic-DMDDO. In addition, wells were incubated with a solution of Pluronic F108. The Pluronic-DMDDO wells were charged with Ni²⁺. A purified hERαLBD solution (that had been dialysed overnight against a PBS buffer in order to remove E2 from the ligand binding domain (LBD) binding sites) was then added to the wells coated with modified and unmodified Pluronic. In addition, uncoated wells were coated with the purified hERαLBD solution. The wells were incubated with the hERαLBD solution. A working solution of 2, 4, 6, 7 ³H-estradiol (³HE2) was then pipetted into each well that contained either modified or unmodified Pluronic, or hERαLBD. The tritiated estradiol working solution was also applied to polystyrene wells that contained no additives. Wells were again washed with distilled water to remove excess radioactively labelled estradiol. Elution buffer (as per wash buffer used in purification procedure containing 500 mM imidazole) was then applied to each well. A volume of 80 μL of each sample was then transferred to individual scintillation tubes. Radioactivity of each sample was then measured via a scintillation counter (Tri-Carb®, 1900A) as counts per minute (CPM) over a period of 5 minutes. A second experiment was conducted using the his6-ARLBD and tritiated testosterone. The experimental parameters were otherwise exactly the same as in the hERαLBD/³HE2 experiment.

Maltose binding protein-hERαLBD fusion protein was immobilized to an amylose functionalized membrane with the amylose present in the membrane surface offering the binding sites for the MBP-hERαLBD immobilization since MBP has an affinity for amylose. The application of the membrane system for the selective recovery of estradiol from solution is also included herein.

Results and Discussion

Membrane Fabrication

Synthetic polymeric membranes were fabricated using the immersion precipitation technique to manufacture nonporous planar and capillary membranes of reproducible physical and chemical composition. The surface chemistries of the membrane polymers were verified using photo acoustic FT-IR analysis. Surface hydrophobicity was calculated using static and dynamic contact angle analysis for the planar and capillary membranes respectively. The candidate membranes chosen in this study add rough surfaces that were inherent to the fabrication conditions use. Membrane surface roughness was, however, found to decrease after surface modification in 5 mg/mL Pluronic F108. The membrane surface hydrophobicity was of the order PVDF>PSU>PEI. Imidazole was used to activate and solubilise EDTA-dianhydride. This yielded a tetra dentate ligand with coordination sites on the octahedral system open for ligand attachment and a non polar centre block available for hydrophobic surface interaction. The ligand Pluronic-DMDDO was characterised using ¹³C NMR spectroscopy and is soluble in water organic solvents and can be stored indefinitely, either in solution or as a desiccate.

A CA/amylose mixture was successfully used as a casting dope for the fabrication of a CA/amylose functionalized membrane using the immersion precipitation technique. The membrane topography and morphology was studied with the aid of the SEM technique, while the surface chemistry of the membrane was monitored with FT-IR. Following the information obtained from the membrane morphology and surface chemistry the membrane could be used for affinity immobilization of the specific bio-ligands MBP and MBP-hERαLBD fusion proteins. Membranes containing higher percentages of amylose were ductile, fragile and therefore could not be used as solid supports for further experimental analyses. Therefore in the present study, only a 2% amylose membrane was used for the immobilization study. In future, however, higher percentages of amylose could be incorporated into the CA membrane but this will be followed by using some plasticizer to render the membrane more resilient to physical damage and deformation.

Receptor Expression

The hERαLBD insert was confirmed to be present in the pET15b as shown via the restrict digestion performed on the isolated plasmid DNA. Analysis via SDS PAGE and Western Blot confirmed the hERαLBD was expressed and was present in the supernatants of both the media which contained additives (E2 and sucrose) and media which contained no additive. Resuspended pellets contained hERαLBD, though most of the hERαLBD remained in solution and thus in the supernatant.

The LBD of the AR, isolated and amplified via PCR, was successfully subcloned into the pTrcHis plasmid. The plasmid construct, designated AR-pTrcHis, was then used to transform TOP10 E. coli. Expression of the histidine tagged ARLBD was confirmed via SDS PAGE and Western Blot analysis.

This study showed that MBP-hERαLBD ligated into pMalc2, and the MBP gene (MalE product) from pMalc2 can be expressed in high yield using the E. coli expression system. Results obtained from SDS PAGE and Western Blot analyses of the two respective cell lysates. A band corresponding to an apparent molecular mass of 66 KDa was in good agreement with the molecular mass of MBP-hERαLBD reported in literature.

Receptor Purification and Antibody Production Against Fusion Receptors

The hERαLBD and ARLBD were purified on an AKTA® protein purification system. The one-step affinity purification system with amylose as the solid phase was used for the effective purification of MBP-hERαLBD proteins. Antibodies were raised against the estrogen bound and unbound forms of the hERαLBD. There is a degree of distinction between the E2 bound and unbound form of hERαLBD at higher serum dilutions (>10⁻²). Sensitivity at lower serum dilutions is diminished (between 10⁻¹ and 10⁻²) and the primary antibodies are unable to distinguish between the two forms of hERαLBD.

Binding Studies

The initial tests were carried out in polystyrene microtitre plates. The Pluronic-DMDDO, charged with hERαLBD, was coated onto the surface of the polystyrene microtitre plate. Two controls used in the experiment included wells that were coated with Pluronic F108 and uncoated wells. A significantly higher binding of radioactive estrogen could be shown in the wells coated with Pluronic-DMDDO charged with hERαLBD when compared to that of the controls (wells coated with Pluronic F108 and only and uncoated wells), indicating immobilization of hERαLBD. The second Pluronic-DMDDO experiment, charged with his6-ARLBD, yielded similar results in that the wells charged with the protein were able to bind a significantly higher amount of radioligand compared to the controls. These experiments clearly indicate proof of concept as it is apparent that not only were the LBDs of the androgen and estrogen receptors immobilized, but the receptors retained bioactivity by binding to androgenic and estrogenic compounds in water solutions, respectively.

Prior to the CA/amylose flat-sheet membrane binding assay, the activity and binding of the recombinant protein, MBP-hERαLBD, onto the resin was tested using ³HE2. For the determination of the E2-binding, a slurry of amylose resin was incubated overnight with extract containing MBP-hERαLBD. The washed slurry was later incubated for 3 h at room temperature with 300 μL of buffer A containing ³HE2 working solution. The washing step was repeated and 4 mL of scintillation cocktail was added to the slurry. The mixtures were transferred into scintillation vials and later counted using a liquid scintillation counter.

It is believed that the disclosed embodiments provide a new detection device for detecting endocrine disrupting compounds and that they have conclusively demonstrated that the affinity immobilisation of estrogenic and androgenic compounds via the LBD of the human estrogen and androgen receptors on inert membrane contactors is possible and practically feasible.

1. A detection device for detecting endocrine disrupting compounds, comprising:
a support structure; and
ligand binding domains (LBDs) of at least one sex hormone receptor, immobilised on the support structure, in which the endocrine disrupting compounds are in the form of at least one of androgenic and estrogenic compounds, wherein the at least one sex hormone receptor is in the form of at least one of an estrogen receptor and an androgen receptor.

2. The detection device as claimed in claim 1, in which the detection device includes a linker molecule between the support structure and the LBDs, for immobilising the LBDs on the support structure.

3. The detection device as claimed in claim 1, in which the sex hormone receptor is in the form of at least one of a human estrogen receptor and a human androgen receptor, having ligand binding domains hERαLBD and hARLBD respectively.

4. The detection device as claimed in claim 3, in which the ligand binding domains include a peptide tag and is in the form of any one of his₆-hERαLBD and his₆-hARLBD.

5. The detection device as claimed in claim 3, in which the ligand binding domain hERαLBD is expressed as a fusion protein and is in the form of maltose binding protein-hERαLBD (MBP-hERαLBD).

6. The detection device as claimed in claim 2, in which the support structure is in the form of a membrane.

7. The detection device as claimed in claim 2, in which the support structure is in the form of a membrane contactor.

8. The detection device as claimed in claim 7, in which the membrane contactor is in the form of a cellulose acetate hybrid membrane.

9. The detection device as claimed in claim 8, in which the cellulose acetate hybrid membrane is in the form of an affinity cellulose acetate-amylose hybrid membrane.

10. The detection device as claimed in claim 9, in which the linker molecule is in the form of maltose binding protein.

11. The detection device as claimed in claim 6, in which the support structure is in the form of an inert membrane.

12. The detection device as claimed in claim 11, in which the inert membrane is in the form of a synthetic polymeric membrane.

13. The detection device as claimed in claim 12, in which the synthetic polymeric membrane is fabricated using an immersion precipitation technique.

14. The detection device as claimed in claim 12, in which the synthetic polymeric membrane is planar and nonporous.

15. The detection device as claimed in claim 12, in which the synthetic polymeric membrane is cast from at least one of polysulphone (PSU), polyether imide (PEI) and polyvinylidene fluoride (PVDF) solutions.

16. The detection device as claimed in claim 15, in which the linker molecule is in the form of a modified poloxamer.

17. The detection device as claimed in claim 16, in which the poloxamer is in the form of a difunctional block copolymer surfactant terminating in primary hydroxyl groups.

18. The detection device as claimed in claim 17, in which the poloxamer is in the form of poly(ethylene glycol)-b/ock-poly(propylene glycol)-block-poly(ethylene glycol) (Pluronic F108).

19. The detection device as claimed in claim 18, in which the modified Pluronic F108 is in the form of Pluronic F108 of which the hydroxyl end groups are modified to a metal chelating moiety that specifically binds Ni-ions, serves as an immobilised metal affinity chromatography matrix, and provides a metal affinity immobilised receptor ligand binding domain.

20. The detection device as claimed in claim 19, in which an ethylene diamine tetraacetic acid dianhydride is coupled to the terminal hydroxyl end groups of Pluronic F108 via a two-step reaction, to create a new metal affinity ligand, Pluronic-N,N dicarboxymethyl-3,6-diazaoctanediote (Pluronic-DMDDO).

21. A method of detecting endocrine disrupting compounds (EDC's), comprising:
contacting a detection device as claimed in claim 1 with a sample to be tested, such that if the sample contains endocrine disrupting compounds, the compounds will bind to the ligand binding domains; and
performing a colorimetric assay to indicate bound endocrine disrupting compounds.

22. The method of detecting endocrine disrupting compounds as claimed in claim 21, in which performing the colorimetric assay includes the steps of:
saturating the ligand binding domains with EDCs, upon which the ligand binding domains change conformation and are activated forming activated EDC-ligand binding domain complexes;
adding monoclonal antibodies, raised against the activated EDC-ligand binding domain complexes, and allowing the monoclonal antibodies to bind to the activated EDC-ligand binding domain complexes,
using the monoclonal antibodies in an enzyme linked immuno-assay; and
observing bound enzymes, which indicate the presence of EDCs in the sample.

23. The method of detecting endocrine disrupting compounds as claimed in claim 21, in which performing the colorimetric assay includes the steps of:
adding enzyme labelled steroid ligands for the specific receptors; and
determining the degree of receptor saturation.

24. The method of detecting endocrine disrupting compounds as claimed in claim 21, in which performing the colorimetric assay includes the steps of:
adding heat shock proteins, which bind and activate bound receptors; and
adding antibodies against the heat shock proteins to indicate bound receptors and thus endocrine disrupting compound binding.
